(19) United States
(12) Patent Application Publication
(10) Pub. No.: US 2004/0005698 A1
(43) Pub. Date: Jan. 8, 2004

(54) Method and apparatus for moving and stirring compost

(76) Inventor: Jean Claude Chenu, rue de Ponte (FR)

Correspondence Address: Pendorf & Cutliff, PO Box 20445, Tampa, FL, 33622-0445, US

(21) Appl. No.: 10/333,808

(22) PCT Filed: Jul. 24, 2001
(86) PCT No.: PCT/FR01/02402

(30) Foreign Application Priority Data
Jul. 24, 2000 (FR) 00/09649

(51) Int. Cl. C12M 1/02

(52) U.S. Cl. 435/290.2; 366/346

(57) ABSTRACT

The invention concerns a method for operating a machine stirring (M) fermentable matter (D) in a fermenting installation. Said method is characterised in that it consists in stirring the fermentable matter (D) by turning it and blowing it to the rear of the machine (M) so as to gradually move it along said passage of a first feeding end (Ce) towards a discharging end (Cs). The invention also concerns the machine (M) consisting of a stirring member (300) comprising a propeller shaft (310) peripherally provided with stirring tools (320) arranged peripherally around said propeller shaft (310) in a pair of reverse pitch turns symmetrically arranged relative to the perpendicular median plane of symmetry of said propeller shaft (310). The invention is useful for producing aerobic fermented mixtures.

### FIELD OF THE INVENTION

This invention deals with the manufacturing of mixtures having been aerobically fermented, better known as compost, and generally used for the improvement of agricultural soil.

### DESCRIPTION OF PRIOR ART

Generally speaking, the transformation of fermentative material into compost is obtained by bioconversion of the material, which having been discharged into reservations (pits, containers, aisles) provided to that effect, is subjected to a degree of moisture and to an oxygenation sufficient to maintain actively alive bacterial micro-organisms that are necessary for the proper development of the fermentation phase itself. The physico-chemical balance of such fermentation is obtained more precisely by alternating stirring phases in order to facilitate the oxygen penetration into the biomass of the fermentative material and quiet periods where fermentation is allowed to develop by generating heat that facilitates the proliferation of micro-organisms and allows for the elimination of most morbific agents. The quality of the compost that is finally obtained is then characterised by its stability in time and its homogeneity and is therefore dependent upon the stirring process having been implemented.

There are in prior art several categories of installations allowing the implementation of a compost manufacturing process from the transformation of fermentative material.

The first category of such installations consists in laying on an outside platform fermentative material under the shape of windrows on which is spanned a self-propelled device, which is pneumatically mounted and provided with a mechanical stirring means, said device turns over said windrows as it is advancing. Such an installation does not allow for the homogenisation of the stirring, which is remaining static, nor does it allow for the control of temperature and degree of fermentation of the biomass of the fermentative material because of unforeseen climatic variations. Moreover, the width of the windrows is restricted as it is limited to the width of the mechanical stirring means that may not obviously exceed the size of the self-propelled device that is allowed on the roads.

A second category of installations consists in laying fermentative material into a pit in which one or several vertical stirring means having the shape of an helix simultaneously raise and lower the fermentative material. Such pits that are limited as far as their volume is concerned, do not provide for a good oxygenation of the stirred fermentative matter.

A third category of installations consists in laying the fermentative material in aisles having a size of roughly tens of meters provided with a plane slot having a wall on each side to be used as rolling path by a stirring device constituted by a horizontal driving rod set at right angles and around which are placed blades regularly spaced such as in a bladed wheel. The loads exerted on such blades are very important as they are concentrated on the extremities of the blades and are transmitted on the whole length of said blades at each impact with the material. Consequently, the driving rod has to support very high stirring loads on aisle widths greater than ten meters. Besides, the effect of such blades is more to turn over the fermentative material on the spot rather than to stir it. It was contemplated to replace said “bladed wheel” by a portal supporting a stirring screw found in the second category of installations with pits and which moves along said portal, so as to ensure a sweeping on the width of the aisle and on the length of the latter due to the step-by-step forward motion of the portal. However, the homogenisation of the stirring is not improved as it is disorganised because a part of the fermentative material goes back to the surface due to the rotary motion of the screw and another part is splashed laterally on the walls of the aisle and backwards due to its sweeping motion.

The various categories of installations described hereabove are all showing important deficiencies at the level of the homogenisation of the stirring as well as at the level of the permanent oxygenation of the fermentative material. Such deficiencies have then a great impact on the length of the fermentation and only allow so far to implement installations manufacturing compost in periods of about six months only. Such a long fermentation duration is leading to large stocks of base material and an offset availability of compost because of the seasonal delivery of fermentative material.

### DESCRIPTION OF THE INVENTION

Taking into account such facts and a pre-established specifications sheet, the applicant led researches that resulted in a new concept of a device allowing for the dynamic stirring of the fermentative material as opposed to the static stirring of prior installations. To that effect, the applicant is proposing a working process for a device that consists in stirring the fermentative material by turning it over and by projecting said matter in the back of the device so as to move it progressively along said aisle from a first delivery extremity towards a second evacuation extremity. Such manner of stirring the fermentative material by creating a continuous and regular motion of said matter each time the device is passing by, allows for the evacuation at one extremity of the aisle of the compost so that it may be recovered and it also allows for the delivery of fermentative material at the other extremity of the installation. Consequently, the fermentative material is stirred and projected backwards by the device and it forms then a ventilated pile, that is permanently oxygenated, which is maintaining the proliferation of micro-organisms.

In order to implement the working process of the invention, the applicant also imagined an original concept for the stirring device for installations manufacturing compost of the third category, that is of the type that stirs the fermentative material laid in at least one fermentation aisle. Such device is classically made of a driving rod, which is provided at its periphery with stirring tools, and which, laid horizontally and at right angles with said aisle, is provided with a rotary motion on itself to ensure the stirring of the fermentative material on one hand, and on the other hand it is provided with a translational motion along said aisle to move from one extremity of said aisle to the other. The original concept of such device is based on the fact that the stirring tools of the stirring element allowing for the stirring of the fermentative material by projecting it in the back of the device, are placed on the periphery of said horizontal driving rod according to at least one pair of reverse pitch turns placed symmetrically with respect to the perpendicular median plane of symmetry of said driving rod.

The tools of each of the turns of a same pair that are placed symmetrically on each side of the perpendicular median plane of said horizontal driving rod, exert as a pair identical loads that are symmetrically distributed on the whole length of said driving rod, which is then subjected to mechanical loads that are perfectly balanced.

According to a particularly advantageous embodiment of the invention, the aforementioned stirring tools of the stirring element are constituted by an assembly of arms mounted individually of the driving rod. Such individual and spiroid placement of the tools around the driving rod allows, for a complete rotation of the driving rod, to play on the frequency of the mechanical impacts on the fermentative material because of the definition of the number of tools placed along each turn and to multiply the pairs of turns acting simultaneously on each side of the vertical median plane of said driving rod because of the definition of the number of pairs of turns. Therefore, such a placement of the stirring tools around said driving rod is offering a sequential and variable distribution of the tools, which by acting on the frequency and the amplitude of the mechanical impact of each tool for a same load of the driving rod, greatly improves the homogenisation of the stirring operation on the whole width of the aisle. Such spiroid organisation of the tools is ensuring through a mechanical effect a better attack of the particles and therefore a superficial continuous grinding and bursting of the fermentative material that increases the exchange surfaces and thus a permanent microbiological reseeding that is very favorable to the bioconversion of the material.

Such improvement in the quality of the stirring of the fermentative material added to a good oxygenation of the biomass, as the lower and upper layers are turned upside down at each passage of the device and the microbiological reseeding is permanent, allows to greatly decrease the fermentation duration, which is reduced from around six months to less than a month.

According to a particularly advantageous embodiment of the invention, the device is constituted by a skeleton, used as logical structure for the functional elements, composed of:

a rolling frame allowing the translational motion of the whole device along two rolling paths provided on the upper edge of two longitudinal walls on each side of the aisle,

and a frame that, supporting said stirring element, is mounted in a movable manner with respect to the first rolling frame in order to allow said stirring element to move from a lower working position where said stirring tools are in contact with the fermentative material and a raised position where said stirring tools are not in contact with the fermentative material, so that the whole device may freely move above said fermentative material. Such kinematics is intended to allow a faster displacement of the device along the aisle without any stirring work of the fermentative material.

According to a first preferred embodiment of the invention, the mobile frame will be mounted in an articulated manner on the first rolling frame around a horizontal support axis carried by the latter perpendicularly to the longitudinal axis of the aisle, so that the aforementioned stirring element will be endowed with a rocking motion around a horizontal axis perpendicular to the longitudinal axis of the aisle.

According to a second preferred embodiment of the invention, the mobile frame will be mounted so as to slide vertically with respect to the first rolling frame, so that the aforementioned stirring element will be endowed with a translational motion along an axis perpendicular to the longitudinal axis of the aisle.

According to another particularly advantageous embodiment of the invention, the rotary motion of the aforementioned driving rod of the stirring element is controlled by strain gauges allowing to regulate the motions of displacement and rotation of the stirring element with respect to the stirring strain provided by the fermentative material against the tools of the aforementioned element. Such automatic control of the device by the stirring load by controlling in particular the intensity of the driving motor of the stirring element may for instance trigger the stopping of the device (to prevent said strain from having repercussions on the whole device and from causing a defaillance) or may implement an original phase of the work process, which will be described later in a more detailed manner and which consists in taking, with a prior backward return of the device, the fermentative material showing a higher resistance to stirring but with a slower progression speed. Likewise, when associated with a digital indicator, such strain gauge may give the user the possibility to adjust the progression speed of his device according to the measured and indicated resistance.

The fundamental concepts of the invention having been set forth hereabove, other details and features will come forth more clearly when reading the following description giving, as a non limiting example and with respect to the attached drawings, an embodiment of a stirring device of an installation manufacturing compost according to the invention.

### SHORT DESCRIPTION OF THE DRAWINGS

FIG. 1 represents a side-face view of such a stirring device in the lowered position.

FIG. 2 represents a side-face view of the device of FIG. 1 in the raised position and/or at rest.

FIG. 3 represents a global view of an element of the device of FIG. 1, ensuring the stirring operation itself.

FIG. 4 represents a side-face view of the device of FIG. 1 in the lowered position and during a stirring cycle.

FIGS. 5a, 5b, 5c, . . . 5l represent schematic side-face views showing the various phases of a to-and-fro working cycle of a device according to the invention in an aisle filled with fermentative material.

FIG. 6 represents a side-face view of the device of FIG. 1 in the lowered position and provided with two optional devices.

### DESCRIPTION OF THE PREFERRED EMBODIMENTS

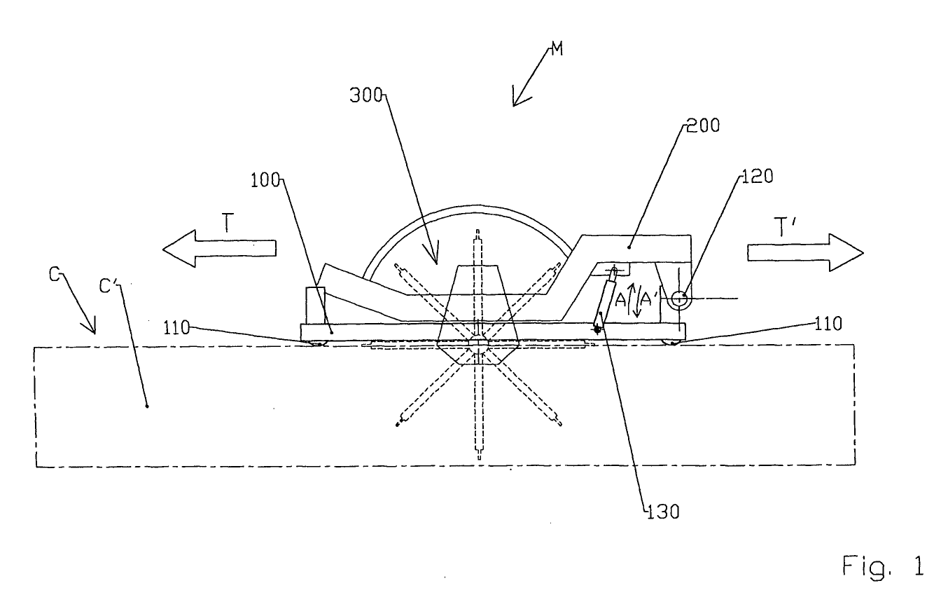

Such as shown in the drawing of FIG. 1, the stirring device globally referenced as M and intended to move above a fermentative material laying in a fermentation aisle C is based on a skeleton obtained from a metallic assembly of sections and mechanical tubes classically welded and previously sanded in order to remove potential rusted points, then treated with respect to the corrosive environment where they will move before being painted. Such skeleton obtained in a classical manner in order to serve as a logical structure to the functional elements of the device M is especially composed of:

a rolling frame 100 allowing the translational motion of the device M (arrows T and T′) along the two rolling paths provided on the upper edge of the two longitudinal walls on each side of the aisle C,

a frame 200 articulated (arrows A and A′) on the first frame 100 around a horizontal axis perpendicular to the longitudinal axis of the aisle C.

The rolling frame 100, moving on two rolling paths defined by two tracks anchored on the upper edges of the two longitudinal walls C′ (one of them only is represented by a dot-and-dash line) on each side of the aisle C, is provided at its four extremities and by means of rest bars, with four rollers 110 independently motorised in order to allow the device M to move along the walls C′ (arrows T and T′) without being unbalanced. Such rolling frame 100, properly fixed on the tracks, is also provided with sensors of limit of travel, which are not shown, in order to limit the displacements T and T′ of the device at the extremities of the aisle C.

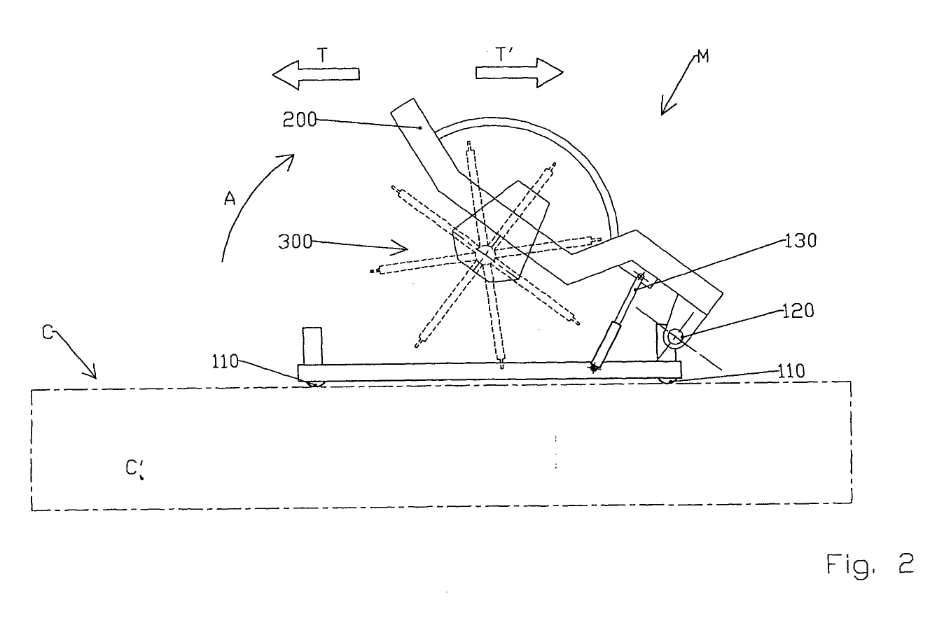

As for the frame 200 shown here in an articulated mode, it supports a stirring element 300 and is mounted in a pivoting manner (arrows A and A′) around a horizontal support axis 120 associated to the rolling frame 100, under the effect of the extension (arrow A) or the retraction (arrow A′) of two raising heavers placed on each side of the device M between the rolling frame 100 and the articulated frame 200. Such articulated frame 200 also allows said stirring element 300 to move in a lowered working position, such as shown on the drawing of FIG. 1, to a raised position, such as shown on the drawing of FIG. 2, in order to allow the whole device M to move freely above said fermentative material laid in the aisle C between the two walls C′.

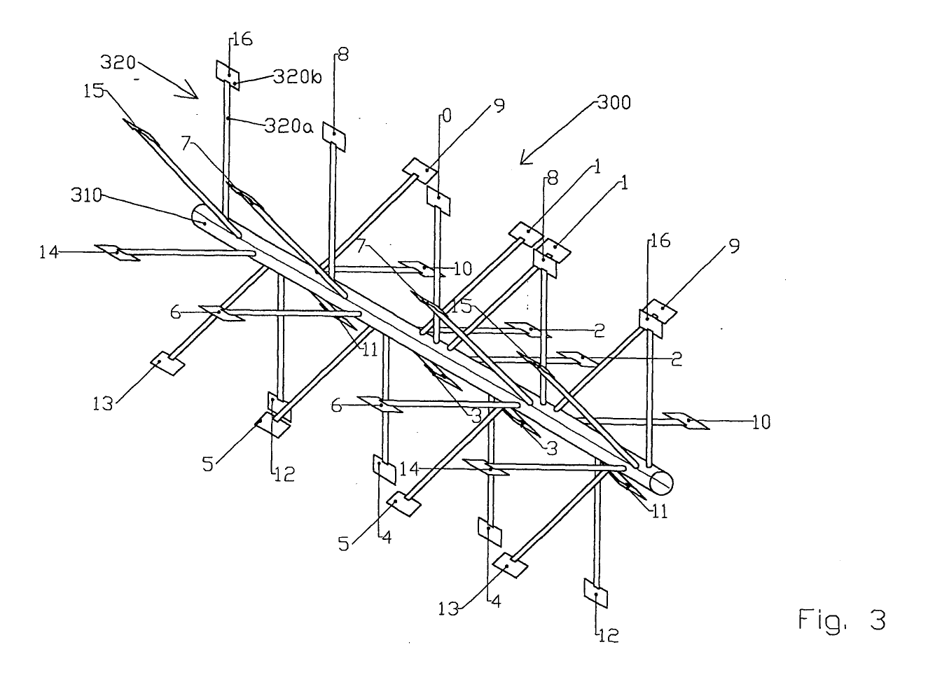

According to the fundamental concept of the invention, the stirring element 300, shown in more details on the drawing of FIG. 3, is composed of a driving rod 310 provided at its periphery with stirring tools 320 placed peripherally according to pairs of reverse pitch turns with respect to the perpendicular median plane of symmetry of said driving rod 310. Thus, with respect to the theoretical vertical median plane of symmetry going through the position 0 of the central tool 320, the other tools 320 are placed symmetrically by pairs in the same positions 1, 2, 3, 4, 5, 6 . . . 16, so that the two pairs of tools n° 8 and n° 16 will always act simultaneously on each side of the median tool n° 0 like the two other pairs of tools n° 2 and n° 10, and so forth for the eight tools of each turn. As it has been explained at the beginning of this description, the advantage of such layout is to distribute in a uniform manner on the whole length of the driving rod 310 the loads exerted on the tools 320. Naturally, the number (2) of pairs of turns and the number (8) of tools for each turn may be adapted to the importance of the device and the volume of fermentative material to be treated.

According to a particularly advantageous feature of the invention, the stirring tools 320 are constituted by individual arms 320a mounted in movable manner (by pins) on the driving rod 310 so that they may be replaced more rapidly.

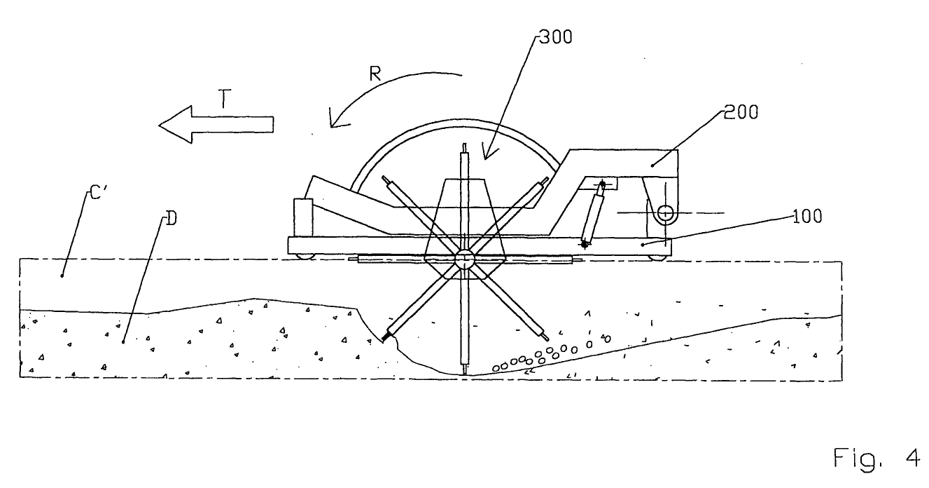

According to another particularly advantageous feature of the invention, the arms 320a of the aforementioned stirring tools 320 are provided with an enlarged extremity 320b forming a blade, the rectangular or trapezoidal shape of which will be adapted in order to facilitate the functions of tangential raking, transportation and projection of the fermentative material in the back of the device M, as shown on the drawing of FIG. 4 where it is represented in a working cycle where the articulated frame 200 is in the lowered position resting on the rolling frame 100, the rolling rollers 110 of the rolling frame 100 slowly move the device in the direction of the arrow T and the stirring element 300 is rotating in the direction of arrow R.

Another goal of the invention is to automatically control the speed of the rotation of the driving rod 310 with the stirring strain of the material opposed to the tools 320. In order to achieve such control, the rotary motion of the aforementioned driving rod is controlled by strain gauges, which control the intensity of the rotary driving motors allowing to stop the rotation of the driving rollers 110 of the rolling frame 100 and to start a new phase of the working cycle of the device M, which consists in having the device M return backwards on a pre-established distance with or without raising the stirring element 300 in order to stir again this section of fermentative material more resistant to grinding, and with a slower progressive speed, for instance.

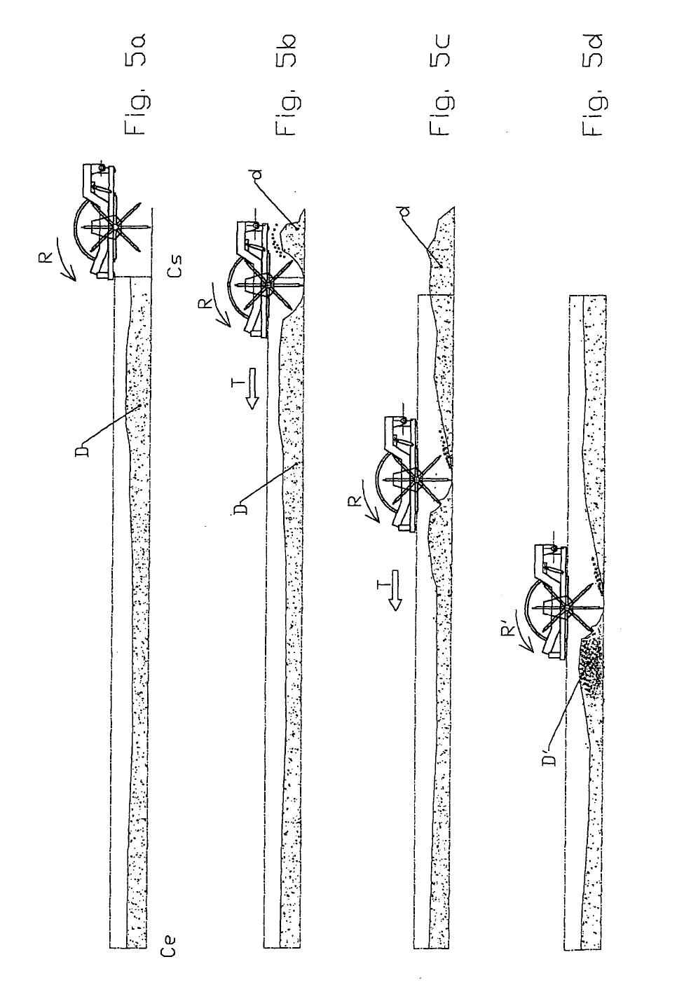

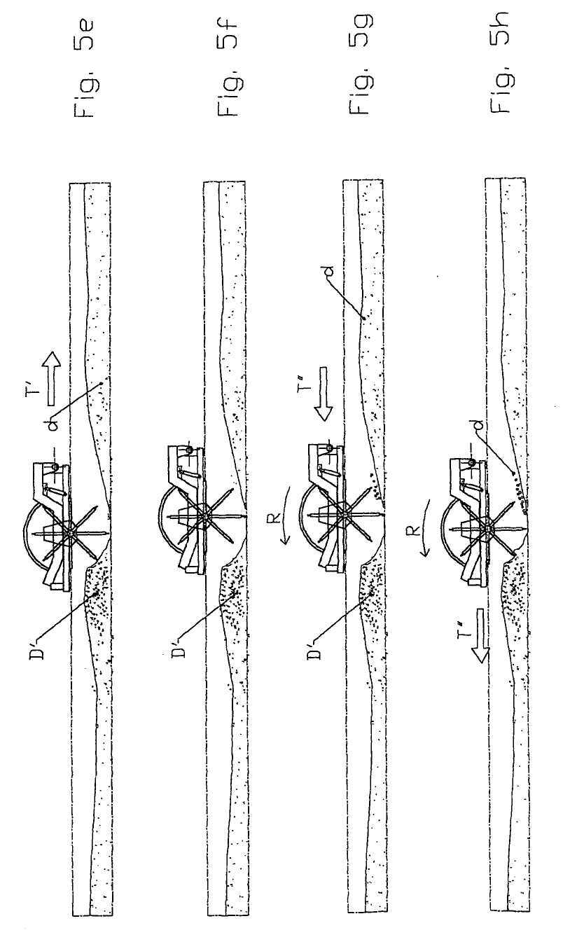

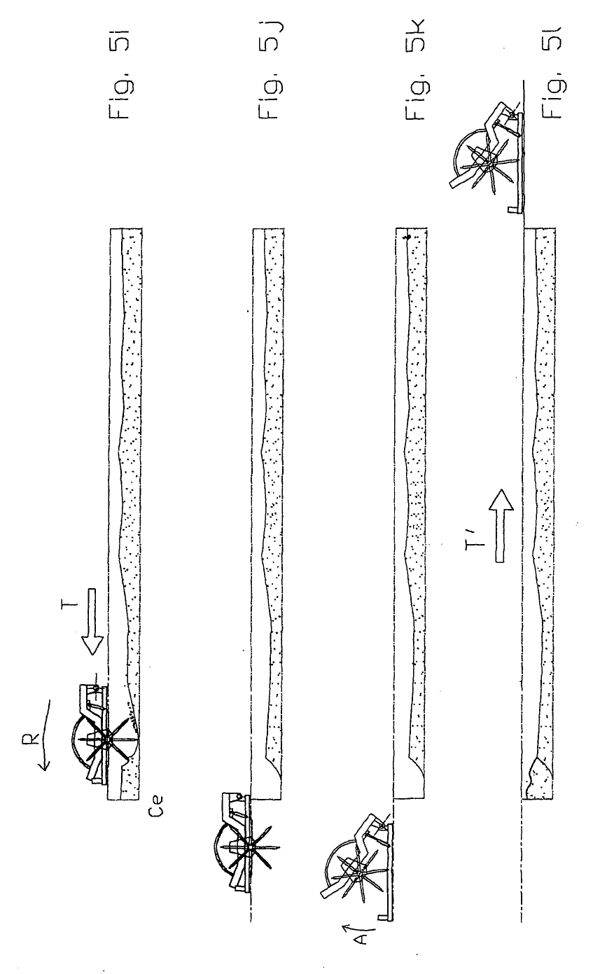

The drawings of FIGS. 5a, 5b, 5d, . . . 5l are intended to illustrate the various phases of a working cycle that may be implemented by the device M, such cycle being given as an illustration and it may receive other parameters easily implement by the man of the art. In order to implement such process, all the displacement motions T, T′ and T″ of the rolling frame 100, the articulation motions A and A′ of the frame 200 and the rotary motions R of the stirring element 300 are controlled by a programmed station mounted on the device M and which, according to the sensors of limit of travel, the strain gauges, the security requirements (thermic and electrical protections) and pre-established instructions, is coordinating the proper course of the following operations.

On the drawing of FIG. 5a showing the beginning of the working cycle, the device M is positioned at one evacuation extremity Cs of the aisle C filled with fermentative material D, with its stirring element 300 lowered and the driving rod 310 displaying a rotary motion R.

On the drawing of FIG 5b, the device M is moving slowly (arrow T) along the aisle C with its stirring element 300 lowered and rotating (arrow R), so that a volume d of the fermentative material that has just been stirred is displaced in the back of the device M. In fact, such volume d of fermentative material that was subject to an identical displacement at each previous passage of the device M and progressively since the delivery extremity of the aisle C where it has been delivered, corresponds to a volume of compost that may be evacuated for recovery. Such volume d may be recovered any time during the motion of the device M towards the delivery extremity Ce of the aisle C.

On the drawing of FIG. 5c, the device M moves (arrow T) along the aisle C with its stirring element 300 lowered and in rotation (arrow R), always displacing volumes d of fermentative material in the back of the device M.

On the drawing of FIG. 5d, where it can be seen that volume d has been evacuated, the tools 310 of the stirring element 300 encounter a volume of fermentative material D′ that is more compact, thus more resistant to stirring, so that the strain opposed to the stirring tools 320, through the strain gauges, is transmitted to a command module of the programmed station that will trigger the stopping of the device M.

On the drawing of FIG. 5e, the device M after having been stopped, moves slightly backwards in the direction of arrow T′, which is opposite to the direction of arrow T, for a pre-programmed duration (about five to ten minutes), at the end of which it stops, as shown on the drawing of FIG. 5f, and starts again forward in the direction of arrow T″, having the same direction as arrow T, and with the stirring element 300 having a rotary motion R (see drawing of FIG. 5g) but with a slower translational speed.

On the drawing of FIG. 5h, the more compact fermentative material D′ does no longer counter the passage of the device M, so that at the end of a pre-programmed duration, the device M moves again at its initial normal speed (arrow T of FIG. 5i) and progresses until the delivery extremity Ce of the aisle C, all the while projecting in the back of the device M volumes of fermentative material d.

On the drawing of FIG. 5j, the device M reaches the delivery extremity Ce of the aisle C, at the end of which the limits of travel trigger the stopping of the rotation of the driving rollers 110 and the rotation R of the driving rod 310.

On the drawing of FIG. 5k, the articulated frame 200 is articulated (arrow A) in the raised position and removing the stirring element 300 from the fermentative material D.

On the drawing of FIG. 5l, the device M is moved rapidly in the direction of arrow T′ until the extremity Cs of the aisle C where limits of travel halt the rotation of the driving rollers 110 and the device M is ready for a new working cycle. As soon as the device M has reached the evacuation extremity Cs, base material may again be delivered to the delivery extremity Ce.

Obviously, the aforementioned process may be adapted, for instance, by ordering to the stirring element 300 a displacement and a rotation that are opposite to those of arrows T and R, so as to stir the fermentative material by displacing it in the two directions in order to delay the evacuation of the compost.

It is understood that the process for manufacturing compost and the stirring device that were described and shown hereabove, were made for publication purposes rather than for limitation purposes. Indeed, various arrangements, modifications and improvements may be brought the above example without, for that matter, going beyond the scope of the invention such as defined in the claims.

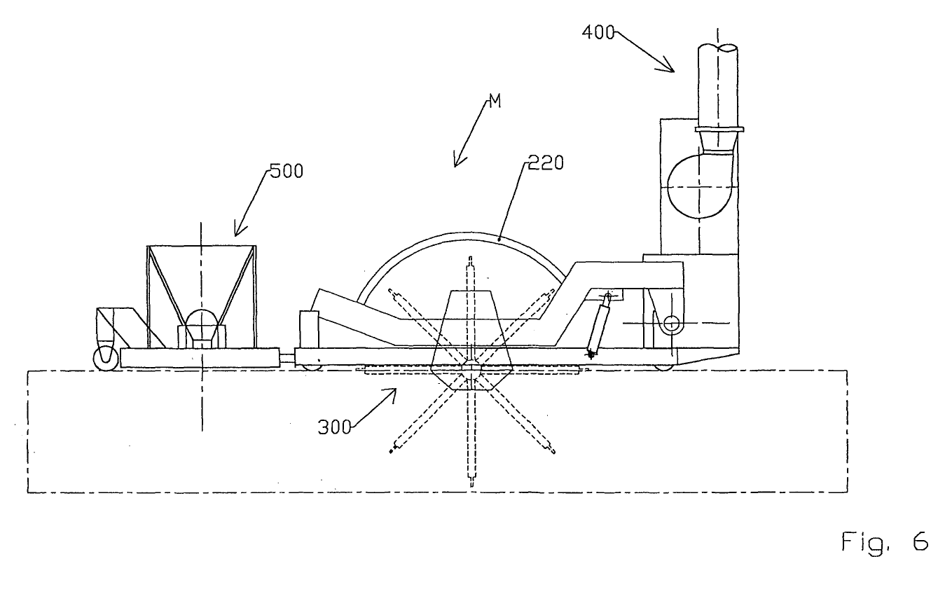

For instance, protective metal sheets or hoods 220 may cover the transmission portion that drives the stirring element 300, in order to avoid any projection of fermentative material upwards and canalise gas releases (ammonia vapors, steam, etc. . . . ) towards a discharging hood 400 that may be properly placed at the back of the device M, as shown on the drawing of FIG. 6.

Likewise, it would also be possible to attach in a movable manner on the front of the device M a mud or liquid delivery hopper 500 (see FIG. 6) provided with an evacuation system activated manually or automatically in order to regulate the degree of moisture of the mass of fermentative material on the whole width and/or length of the aisle C.

1. Working process of a device (M) stirring fermentative material (D) of a fermentation installation, wherein the fermentative material is stirred by turning it over and by projecting it in the back of the device (M), so that it is progressively displaced along said aisle (C) of a first delivery extremity (Ce) towards a second evacuation extremity (Cs).

2. Working process according to claim 1, wherein said device (M) moves translationally (arrow T) along the aisle (C), while simultaneously ensuring the stirring of the fermentative material (D).

3. Device allowing the implementation of the working process according to claims 1 and 2 ensuring the stirring of fermentative material (D) laid in at least one fermentation aisle (C) of a composting installation, where a stirring element (300) composed of a driving rod (310) provided with stirring tools (320) at its periphery and which, placed horizontally and at right angles with said aisle (C), displays a rotary motion on itself (arrow R) in order to ensure the stirring of the fermentative material (D) on one hand, and on the other hand displays a translational motion (arrow T, T′ or T″) along said aisle (C) in order to move from one extremity (Ce) to the other (Cs) of the latter, wherein the stirring tools (320) of the stirring element (300) allowing for the stirring of the fermentative material (D) by projecting it in the back of the device (M), are placed at the periphery of said driving rod (310) according to at least one pair of reverse pitch turns placed symmetrically with respect to the perpendicular median plane of symmetry of said driving rod (310).

4. Device according to claim 3, wherein it is constituted by a skeleton, which is used as logical structure for the functional elements, and which is composed of:
a rolling frame (100) allowing the whole device (M) to move in translation along two rolling paths located on the upper edge of the two longitudinal walls (C′) on each side of the aisle (C),
and a frame (200) that, supporting said stirring element (300), is mounted in a mobile manner (arrows A and A′) with respect to the first rolling frame (100) in order to allow said stirring element (300) to move from a lowered working position where said stirring tools (320) are in contact with the fermentative material (D) to a raised position where said stirring tools (320) are not in contact with the fermentative material (D), so that the whole device (M) may freely move (arrows T and T′) above said fermentative material (D).

5. Device according to claim 1, wherein the aforementioned stirring tools (320) of the stirring element (300) are constituted by an assembly of arms mounted individually on the driving rod (310).

6. Device according to claim 3, wherein the rotary motion (arrow R) of the aforementioned driving rod (310) of the stirring element (300) is automatically controlled by strain gauges allowing for the regulation of the displacement (arrows T and T′) and/or rotary (arrow R) motions of the stirring element (300) with respect to the stirring strain displayed by the fermentative material (D′) against the tools (320) of the aforementioned element (300).

7. Device according to claims 3 and 4, wherein the aforementioned movable frame (200) is articulated (arrows A and A′) on the first rolling frame (100) around a horizontal support axis (120) carried by the latter perpendicularly to the longitudinal axis of the aisle (C), so that the aforementioned stirring element (300) is rocking back and forth (arrow A or A′) around a horizontal axis (120) perpendicular to the longitudinal axis of the aisle (C).

8. Device according to claims 3 and 4, wherein the aforementioned mobile frame (200) is mounted so as to slide vertically with respect to the first rolling frame (100), so that the aforementioned stirring element (300) displays a translational motion along an axis perpendicular to the longitudinal axis of the aisle (C).

9. Device according to claims 3 and 5, wherein the arms (320a) of the aforementioned stirring tools (320) of the stirring element (300) are mounted in a movable manner on said driving rod (310).

10. Device according to claims 3 and 5, wherein the arms (320a) of the aforementioned stirring tools (320) are provided with an enlarged extremity (320b) forming a blade.
